(19) United States
(12) Patent Application Publication
(10) Pub. No.: US 2016/0143873 A1
(43) Pub. Date: May 26, 2016

(54) GEL COMPOSITIONS

(71) Applicant: Leo Laboratories Limited, Dublin (IE)

(72) Inventors: Per-Ola Arvidsson, Ballerup (DK); Edit Farkas, Ballerup (DK); Chinar S. Saeed, Ballerup (DK); Mikkel Krogh-Madsen, Ballerup (DK); Karsten Petersson, Ballerup (DK)

(21) Appl. No.: 14/738,753

(22) Filed: Jun. 12, 2015

Related U.S. Application Data
(63) Continuation of application No. PCT/EP2013/076184, filed on Dec. 11, 2013.

(30) Foreign Application Priority Data
Dec. 12, 2012 (GB) GB1222403.6

(51) Int. Cl. A61K 31/22; A61K 9/00; A61K 9/06

(52) U.S. Cl. CPC A61K 31/22; A61K 9/06; A61K 9/0014

(57) ABSTRACT

Substantially anhydrous topical gel compositions comprising ingenol-3-angelate as a suspension in non-aqueous carriers.

### TECHNICAL FIELD

The invention relates to a topical gel composition comprising ingenol-3-angelate as a pharmacologically active agent.

### BACKGROUND OF THE INVENTION

PICATO® is an aqueous gel formulation comprising ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester, also known as ingenol-3-mebutate or PEP005) at a strength of 0.015% or 0.05%. PICATO® was granted regulatory approval in 2012 by the FDA for the topical treatment of actinic keratosis.

The compound ingenol-3-angelate (PEP005) [Sayed, M. D. et. al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7,449,492.

Pharmaceutical formulation of the compound has been described in WO2007/068963, which discloses various gel formulations for the treatment of skin cancer.

Angelic acid and angelic acid esters such as ingenol-3-angelate are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563].

Furthermore, ingenol-3-acylates are known to be unstable as they rearrange to afford the ingenol-5-acylates and ingenol-20-acylates [Sorg, B. et. al, Z. Natuiforsch., (1982), 37B, 748-756].

It is an object of the invention to provide a composition of ingenol-3-angelate which is stable at room temperature for extended periods.

It is a further object of the invention to provide a composition exhibiting favourable penetration characteristics and biological activity.

It is a further object of the invention to provide a composition with reduced skin irritation, favourable cosmetic properties and improved patient compliance.

A crystalline form of ingenol-3-angelate has been described in WO2011/128780. It is also an object of the invention to utilise the properties of the crystalline structure.

### STATEMENTS OF INVENTION

The present invention relates to a substantially anhydrous topical gel composition comprising ingenol-3-angelate as a suspension in a non-aqueous carrier. In some embodiments, the composition may include ingenol-3-angelate as a non-crystalline suspension. In some embodiments, the composition may include ingenol-3-angelate as a crystalline suspension. The ingenol-3-angelate in the composition is usefully present at 0.015% by weight or 0.05% by weight.

The present invention also relates to a substantially anhydrous topical gel composition comprising ingenol-3-angelate as a suspension in a non-aqueous carrier, wherein the composition is not a substantially anhydrous pharmaceutical gel composition for cutaneous application comprising ingenol-3-angelate in dissolved or solubilized form, an acidifying compound, a co-solvent, a viscosity-increasing ingredient, a non-aqueous carrier and a solvent mixture of
(a) a hydrophilic non-ionic surfactant;
(b) a lipophilic non-ionic co-surfactant; and
(c) an oil.

The present invention also relates to a substantially anhydrous pharmaceutical gel composition comprising ingenol-3-angelate as a suspension in a non-aqueous carrier, wherein the composition does not consist essentially of ingenol-3-angelate, benzyl alcohol, polyoxypropylene-15-stearyl ether, liquid paraffin and fumed silica.

The present invention also relates to a substantially anhydrous pharmaceutical gel composition comprising ingenol-3-angelate as a suspension in a non-aqueous carrier, wherein the composition does not consist essentially of:
(i) ingenol-3-angelate, benzyl alcohol, polyoxypropylene-15-stearyl ether, liquid paraffin and Aerosil 200P; or
(ii) ingenol-3-angelate, benzyl alcohol, polyoxypropylene-15-stearyl ether, isopropanol, liquid paraffin and Aerosil 200P.

The present invention further relates to methods for treating a dermal disease or condition comprising topical administration of a gel of the invention.

### BRIEF DESCRIPTION OF DRAWINGS

FIG. 1 shows the percentage of applied ingenol-3-angelate which penetrates into the viable epidermis and dermis (dark shading) and the percentage of applied ingenol-3-angelate which permeates to receptor fluid (light shading) according to the in vitro diffusion test for composition series 11, formulation 03A and PICATO® at the same strength of ingenol-3-angelate by weight of the composition.

### DETAILED DESCRIPTION OF THE INVENTION

The present composition has been found to result in a satisfactory chemical stability of ingenol-3-angelate permitting the composition to be stored at room temperature (25° C.) for extended periods. The satisfactory stability may partly be ascribed to the substantial absence of water in the composition. Moreover, substantially anhydrous topical gel compositions including ingenol-3-angelate as a suspension according to the present invention have been found to reduce the extent of rearrangement of the ingenol-3-angelate, thereby improving stability of the composition.

Human skin, in particular the outer layer, the stratum corneum, provides an effective barrier against penetration of microbial pathogens and toxic chemicals. While this property of skin is generally beneficial, it complicates the dermal administration of pharmaceuticals in that a substantial amount of an active ingredient applied on the skin of a patient suffering from a dermal disease may not penetrate into the viable layers of the skin (the dermis and epidermis) where it exerts its activity. To ensure an adequate penetration of the active ingredient to the dermis and epidermis, it has been preferred to include ingenol-3-angelate in a dissolved state. In previous formulations, the active ingredient has typically been dissolved in an alcohol, e.g. ethanol or isopropanol. However, when used on their own as solvents, alcohols such as isopropanol and diols may give rise to significant skin irritation as they tend to dry out the skin. The gel compositions of the present invention may provide advantageous penetration properties whilst reducing the likelihood of skin irritation.

### Gels

Gels are semisolid dosage forms that contain an agent (a gelling agent) to provide stiffness to a solution or a colloidal dispersion. Gels do not flow at low shear stress and generally exhibit plastic flow behaviour. The gel compositions of the present invention contain suspended particles, and can therefore be classified as a two-phase system. The gel compositions of the present invention could be oleogels/organogels (but not hydrogels).

Whether or not a system behaves as a gel (i.e. exhibits semisolid characteristics, rather than acting as a liquid or solid, etc.) will depend on the various components used within the system and the relative ratios of the different components. It may also depend on the method by which the components that make up the system are mixed, e.g. the order in which the various components are introduced to each other. It is therefore possible for an agent to act as a gelling agent in one environment but not in another. The ability to test compositions to confirm that they are gels as defined herein is within the knowledge of the skilled person in view of the present disclosure and common general knowledge in the field.

The viscosity of a gel can depend on temperature. At low temperatures (e.g. 2-8° C.) the viscosity can be relatively high, but after applying a gel composition of the invention to the skin it can become less viscous because of the combination of increased temperature and the physical stress while being applied. This shear-thinning characteristic gives a gel which is easily distributed on the skin.

In order to effect formation of a gel, it is necessary to have an agent in the composition which acts as a gelling agent. The amount of the gelling agent (or gelling agents, in embodiments where two or more gelling agents are used) required to form a gel will vary on the components within the particular composition. It is common (although not required) to select two or more components which, when used together in particular amounts, effect formation of a gel. These components would typically include a non-aqueous carrier and a viscosity increasing ingredient. For example, when a first non-aqueous carrier of low viscosity (e.g. a liquid) is used as the base of the composition (e.g. in an amount of above about 75% by weight of the composition), it may be necessary to add a viscosity increasing ingredient (e.g. in an amount of about 20% by weight of the composition) in order to effect formation of a gel.

In some embodiments the gel compositions are colourless. In other embodiments they include a coloured substance, which can make it easy to see where the gel has been applied.

Gel compositions of the invention are usually transparent, except for the suspended ingenol-3-angelate solids. In other less preferred embodiments, the gel compositions are turbid in appearance, such that the suspended ingenol-3-angelate solids might not be visible within the gel.

The gel compositions of the invention are typically acidic, because it has been found that alkaline conditions (or even insufficiently strong acidic conditions) may contribute to degradation of ingenol-3-angelate within the gel composition. This means that the gel compositions have sufficient protons for the ingenol-3-angelate to remain stable at room temperature (25° C.) for extended periods, e.g. for 2 years. The gel compositions are substantially anhydrous, so standard pH measurements do not apply. For reference, however, the acidity of gel compositions of the invention should correspond to an aqueous pH of less than about 4.5.

In general, gels are non-invasive and have a localized effect with minimum side effects. Gel compositions of the invention should be suitable for human topical administration. Thus the compositions have the appropriate physical characteristics of topical gels. For instance, the gel compositions have good spreadability, i.e. the gels can readily be spread (e.g. using fingers) after application to the skin to provide a uniform layer. The gel compositions also have excellent extrudability. These properties mean that the gel compositions of the invention are particularly suitable for topical administration. In some embodiments, the gel compositions are applied topically and do not leave a visible residue. The volatile components of the gel compositions may also substantially evaporate to dryness after a certain period of time following topical application. Preferably, the volatile components of the gel composition will evaporate after a therapeutically effective amount of the ingenol-3-angelate has penetrated into the skin (e.g. after about 1 minute, 2 minutes, 5 minutes, 10 minutes, 20 minutes, etc. following topical administration to a subject).

Ingenol-3-angelate

The composition of the invention includes ingenol-3-angelate. Typically, the composition includes ingenol-3-angelate in an amount of from about 0.001% to about 0.5% by weight of the composition. The composition may include ingenol-3-angelate in an amount of about 0.0005%, 0.001%, 0.0025%, 0.005%, 0.01%, 0.015%, 0.025%, 0.05%, 0.075%, 0.1%, 0.125%, 0.15%, 0.2%, 0.25% or 0.5% by weight of the composition. In two particularly preferred embodiments the composition includes ingenol-3-angelate in an amount of 0.05% or 0.015% by weight of the composition.

The compositions of the present invention can be manufactured, for example, from micro- or nano-processed solid state ingenol-3-angelate. Such micro- or nano-processed solid state ingenol-3-angelate can be produced using various techniques, such as micronization (e.g. by ball mill grinding), nanoprocessing (e.g. by grinding), high pressure homogenization and microfluidization.

Ingenol angelate exists in three isoforms: ingenol-3-angelate (isoform ‘b’), ingenol-5-angelate (isoform ‘a’) and ingenol-20-angelate (isoform ‘c’). The compositions of the present invention include ingenol-3-angelate, i.e. isoform ‘b’, which tends to undergo rearrangement to isoform ‘a’ and subsequently to isoform ‘c’. Preferably, the composition includes less than about 1%, and even more preferably less than about 0.5%, of the ‘a’ isoform after a period of 3 months at room temperature (25° C.). Preferably, the composition includes less than about 1%, and even more preferably less than about 0.5%, of the ‘c’ isoform after a period of 3 months at room temperature (25° C.).

In some embodiments, the compositions of the invention include crystalline ingenol-3-angelate. In certain embodiments, the compositions of the invention include crystalline ingenol-3-angelate in which the crystalline form is not a solvate. In certain embodiments, the compositions of the invention include crystalline ingenol-3-angelate in which the crystalline form is orthorhombic. In certain embodiments, the compositions of the invention include crystalline ingenol-3-angelate in which the crystalline form is characterized by an FTIR-ATR spectrum exhibiting attenuated total reflectance peaks at approximately 3535, 2951, 1712, 1456, 1378, 1246, 1133, 1028 and/or 956 cm⁻¹ (±3 cm⁻¹). In certain embodiments, the compositions of the invention include crystalline ingenol-3-angelate in which the crystalline form has a differential scanning calorimetry curve comprising an event with an onset at about 153±about 5° C. Preferably, when the compositions of the invention include crystalline ingenol-3-angelate, the ingenol-3-angelate has a polymorphic purity of at least about 80%, such as about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the compositions of the invention include amorphous (non-crystalline) ingenol-3-angelate. The compositions can include a mixture of amorphous and crystalline ingenol-3-angelate.

The gel compositions of the invention are suspensions, i.e. homogeneous mixtures containing solid particles. The gel compositions may therefore also be described as particulate gels. Therefore, the compositions of the invention include solid ingenol-3-angelate within the gel. However, the compositions may also include dissolved ingenol-3-angelate. The amount of dissolved ingenol-3-angelate may vary from about 1% to about 99% by weight of the total amount of ingenol-3-angelate in the gel. Typically, about 20% to about 25% of the total ingenol-3-angelate is dissolved within the gel. For example, the compositions may be supersaturated.

### Non-Aqueous Carrier

The compositions of the invention include a pharmaceutically acceptable non-aqueous carrier. The non-aqueous carrier functions as a vehicle for the ingenol-3-angelate, and the ingenol-3-angelate is typically dispersed throughout the carrier. Typically, the compositions of the invention include more than one non-aqueous carrier, e.g. two, three, four or five non-aqueous carriers. The one or more non-aqueous carriers are typically present in the compositions in a combined amount of from about 40% to about 99.95% by weight of the composition.

In some embodiments, the non-aqueous carrier can act as an occlusive agent, e.g. it can form a layer on the surface of the skin on application of the composition. This layer can form a hydration barrier sufficient to result in reduction of trans-epidermal water loss, thereby improving in skin hydration.

The non-aqueous carrier may be selected from a mineral oil (e.g. liquid paraffin) or a hydrocarbon or mixture of hydrocarbons with chain lengths ranging from C₅ to C₆₀. The non-aqueous carrier may be petrolatum or white soft paraffin. Such a mixture is usually composed of hydrocarbons of different chain lengths peaking at about C₄₀₋₄₄. The non-aqueous carrier may comprise a mixture of petrolatum and liquid paraffin. Such a mixture may consist of hydrocarbons of different chain lengths peaking at C₂₈₋₄₀.

While petrolatum provides occlusion of the treated skin surface, reducing transdermal loss of water and potentiating the therapeutic effect of the active ingredient in the composition, it tends to have a greasy and/or tacky feel which persists for quite some time after application, and it is not easily spreadable on the skin. It may therefore be preferred to employ paraffins consisting of hydrocarbons of a somewhat lower chain length, e.g. paraffins comprising hydrocarbons with chain lengths peaking at C₁₄₋₁₆, C₁₈₋₂₂, C₂₀₋₂₂, C₂₀₋₂₆ or mixtures thereof. The hydrocarbon composition of the paraffins can be determined using gas chromatography. It has been found that paraffins comprising hydrocarbons with chain lengths peaking at C₁₄₋₁₆, C₁₈₋₂₂, C₂₀₋₂₂, C₂₀₋₂₆ or mixtures thereof are more cosmetically acceptable because they are less tacky and/or greasy on application and more easily spreadable. They are therefore expected to result in improved patient compliance. Suitable paraffins of this type, which are generally termed petrolatum jelly, are manufactured by Sonneborn and marketed under the trade name Sonnecone. In preferred embodiments of the invention the non-aqueous carrier is selected from Sonnecone CM, Sonnecone DM1, Sonnecone DM2 and Sonnecone HV. These paraffins are further disclosed and characterized in WO 2008/141078 which is incorporated herein by reference.

In some embodiments the non-aqueous carrier is an iso-paraffin, e.g. isohexadecane or squalane.

The non-aqueous carrier may also be a silicone. However, the present invention excludes the two silicone-containing compositions disclosed in WO2007/068963.

In some embodiments, the silicone is cyclic, e.g. cyclomethicone. In other embodiments, the silicone can be linear. In other embodiments, the silicone may be branched. Silicones such as cyclomethicone and dimethicone may be used to reduce the viscosity of the composition, for example in embodiments which also include a silicone of higher viscosity.

In some embodiments, the silicone is a solid mixture of stearoxytrimethylsilane and stearyl alcohol, such as that available under the trade name Dow Corning® Silky Wax 10. In some embodiments, the silicone is a mixture of high molecular weight silicone elastomer (12%) and decamethylcyclopentasiloxane (i.e. a cyclopentasiloxane and dimethicone crosspolymer), such as that available under the trade name Dow Corning® ST-Elastomer 10. In other embodiments, the silicone is comprised of a volatile polydimethylcyclosiloxane composed mainly of decamethylcyclopentasiloxane, such as that available under the trade name Dow Corning® ST cyclomethicone (5-NF). Dow Corning® ST cyclomethicone (5-NF) is particularly useful when the composition comprises a further silicone of higher viscosity, such as Dow Corning® ST-Elastomer 10. In some embodiments, the silicone comprises a cyclopentasiloxane and polyoxyethylene/polyoxypropylene dimethicone, such as that available under the trade name Dow Corning® BY 11-030. In some embodiments the composition includes more than one silicone non-aqueous carrier, e.g. two or three silicones.

The non-aqueous carrier may also be an oily solvent. In one embodiment, the oily solvent may be a C₆₋₂₂ acylglyceride, where C₆₋₂₂ acylglyceride means a triglyceride or a mixture of mono- and diglycerides or mono-, di- and triglycerides of C₆₋₂₂ fatty acids, where C₆₋₂₂ acylglyceride means a triglyceride or a mixture of mono- and diglycerides or mono-, di- and triglycerides of C₆₋₂₂ fatty acids. For example, the oily solvent may be a vegetable oil (e.g. sesame oil, sunflower oil, palm kernel oil, corn oil, safflower oil, olive oil, avocado oil, jojoba oil, almond oil, canola oil, coconut oil, cottonseed oil, peanut oil, soybean oil, wheat germ oil, grape kernel oil etc.), or highly purified vegetable oil (e.g. medium chain triglycerides, long chain triglycerides, castor oil, caprylic/capric mono- and diglycerides, caprylic/capric mono-, di- and triglycerides, etc.). Medium chain triglycerides are triglyceride esters of fatty acids with a chain length of 6-12 carbon atoms. A preferred medium chain triglyceride is a mixture of caprylic (C₈) and capric (C₁₀) triglycerides, e.g. available under the trade name Miglyol 810 or Miglyol 812 (which differ only in C₈/C₁₀-ratio, with Miglyol 810 having a lower C₁₀ content). Other particularly suitable oily solvents include fatty acid glycerol polyglycol esters, e.g. available under the trade name Cremophor RH40. Particularly suitable caprylic/capric glycerides are available under the trade name Akoline MCM.

In another embodiment, the oily solvent may be a synthetic oil such as a fatty alcohol ester of a C₁₀₋₁₈ alkanoic acid (e.g. isopropyl myristate, isopropyl palmitate, isopropyl linoleate, isopropyl monooleate and isostearyl isostearate etc.).

In another embodiment, the oily solvent may be a polyoxypropylene fatty alkyl ether (e.g. polyoxypropylene-15-stearyl ether, polyoxypropylene-11-stearyl ether, polyoxypropylene-14-butyl ether, polyoxypropylene-10-cetyl ether or polyoxypropylene-3-myristyl ether etc.). The oily solvent may be a stearyl ether such as that available under the trade name Arlamol® E.

The oily solvent may be an alkyl or dialkyl ester such as ethyl oleate, diisopropyl adipate or cetearyl octanoate. The oily solvent may also be a mono- or diglyceride such as glyceryl monooleate, or a fatty alcohol such as oleyl alcohol.

In some embodiments the composition may include a mixture of two oily solvents, or optionally three oily solvents.

### Viscosity-Increasing Ingredient

The gel compositions may include a viscosity-increasing ingredient. For example, when the composition has a liquid base (e.g. a first non-aqueous carrier which is a liquid and is present in an amount of e.g. above about 75% by weight of the composition), it may be necessary to add one or more viscosity-increasing ingredients (e.g. in an amount of e.g. about 20% by weight of the composition) in order to form a gel. The viscosity-increasing ingredient may therefore function as the gelling agent. However, if the composition has a base of higher viscosity (e.g. a first non-aqueous carrier which is white soft paraffin and is present in an amount of e.g. above about 75% by weight of the composition), there may be no requirement for the additional of a viscosity-increasing ingredient, because the white soft paraffin is capable of acting as a gelling agent.

The viscosity-increasing ingredient can be a wax. The wax may be a mineral wax composed of a mixture of high molecular weight hydrocarbons (e.g. saturated C₃₅₋₇₀ alkanes), such as microcrystalline wax. Alternatively, the wax may be a vegetable or animal wax (e.g. esters of C₁₄₋₃₂ fatty acids and C₁₄₋₃₂ fatty alcohols), such as beeswax or hydrogenated castor oil. In some preferred embodiments the viscosity-increasing ingredient is an inorganic substance such as fumed silica (e.g. available under the trade name Aerosil®, such as Aerosil® 200P, which is a high purity amorphous anhydrous colloidal silicon dioxide). The viscosity-increasing ingredient may also be selected from magnesium stearate, aluminium stearate, a sterol such as cholesterol, a long-chain saturated fatty alcohol such as cetostearyl alcohol. In some preferred embodiments the viscosity-increasing ingredient is a silicone rubber or wax, such as Dow Corning® ST-Elastomer 10 or Dow Corning® Silky Wax 10. Dow Corning® ST-Elastomer 10 and/or Aerosil® are particularly preferred. The composition may include more than one viscosity-increasing ingredient, such as two or three viscosity-increasing ingredients. The viscosity-increasing ingredient may be a mixture of acrylamide acryloyldimethyl taurate copolymer, isohexadecane and polysorbate 80, such as that available under the trade name SEPINEO P600. The viscosity-increasing ingredient may be an alkylpolyglucoside, such as that available under the trade name SEPINEO SE68.

The amount of viscosity-increasing ingredient may vary (according to the viscosifying power of the ingredient), but the composition may include from about 0.5% to about 50% viscosity-increasing ingredient by weight of the composition. When the viscosity-increasing ingredient is microcrystalline wax it is typically present in an amount of from about 0.5% to about 30% by weight of the composition. Where the viscosity-increasing ingredient is SEPINEO P600, it is typically included in an amount of from about 1% to about 10% by weight of the composition, e.g. about 2.5% by weight of the composition. Where the viscosity-increasing ingredient is SEPINEO SE68, it is typically included in an amount of from about 2% to about 30% by weight of the composition, e.g. about 5% by weight of the composition. Where the viscosity-increasing ingredient is Dow Corning® ST-Elastomer 10 and/or Aerosil®, it is typically included in an amount of from about 10% to about 50% by weight of the composition, e.g. 10%, 15%, 20%, 25%, 30%, 35%, 45% or 50% by weight of the composition.

### Co-Solvent

In some embodiments, the composition may include a co-solvent selected from the group consisting of lower alcohols, such as n-propanol, isopropanol, n-butanol, 2-butanol and benzyl alcohol, and diols such as propylene glycol. This may be preferred where dispersion of the ingenol-3-angelate is problematic. These co-solvents may also act as a penetration enhancer aiding the penetration of the ingenol-3-angelate into the skin. Addition of a co-solvent may result in an improved physical stability of the composition. The composition may include more than one co-solvent, e.g. two or three co-solvents. For example, the composition may include benzyl alcohol and isopropanol.

The co-solvent may be present in an amount of from about 0.5% to about 20% by weight of the composition, such as from about 1% to about 15%, e.g. about 1.5%, about 2%, about 2.5%, about 3%, or about 5% by weight of the composition.

### Penetration Enhancer

In some embodiments where the gel composition would otherwise have unsatisfactory penetration characteristics, it may be useful to include one or more penetration enhancers. Typical penetration enhancers include propylene carbonate, transcutol, pyrrolidones such as N-methylpyrrolidone or N-hydroxyalkylpyrrolidone, azone, menthol, eucalyptol, nicotinamide, glycerol, mono-di- or polyglycols, ethylacetate or Eugenol. A particularly preferred penetration enhancer is α-tocopherol.

In one embodiment, the composition includes a penetration enhancer in an amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

In one embodiment, the co-solvent (which may function as a penetration enhancer) and a further penetration enhancer are both present in a combined amount of from about 0.01% to about 20% by weight of the composition, such as from about 0.1% to about 15%, e.g. about 0.1%, about 0.5%, about 1%, about 1.5%, about 2%, about 2.5%, about 3%, or about 5% by weight of the composition.

### Acidifying Compound

The composition of the invention may include an acidifying compound, for example where the stability of the gel composition would otherwise be unsatisfactory. An acidifying compound is a compound capable of providing a net overall acidic environment to the composition which, as outlined above, means that the gel compositions have sufficient protons for the ingenol-3-angelate to remain stable at room temperature (25° C.) for extended periods, e.g. for 2 years. Because the gel compositions are substantially anhydrous, standard pH measurements do not apply, but the acidifying compounds described herein are compounds which give an acidity to the gel composition corresponding to an aqueous pH of less than about 4.5. This pH reduces the tendency of the ingenol-3-angelate to degrade to form the tiglate ester, which typically occurs in more basic conditions.

The composition may include more than one acidifying compounds, for instance it may include two or three acidifying compounds. The acidifying compound may be present in an amount of from about which may be included in the composition in an amount of from about 0.5% to about 10% by weight of the composition, such as from about 5% to about 9% by weight of the composition. In some embodiments, the one or more non-aqueous carriers may act as an acidifying compound. The acidifying compound may be fumed silica, which may be included in the composition in an amount of from about 3% to about 10% by weight of the composition, such as from about 5% to about 9% by weight of the composition. Alternatively, the acidifying compound may be a fatty acid such as oleic acid, lactic acid, linoleic acid, stearic acid, lauric acid, palmitic acid, capric acid, caprylic acid, pelargonic acid, adipic acid, sebacic acid or enanthic acid. The fatty acid is typically present in an amount of from about 0.5% to about 5% by weight of the composition.

### Surfactants

In certain embodiments of the invention the composition includes a hydrophilic non-ionic surfactant and/or a lipophilic non-ionic surfactant.

The term “hydrophilic surfactant” means an oil-in-water surfactant with a hydrophile-lipophile balance (HLB) value of 9-18, and “lipophilic surfactant” means a water-in-oil surfactant with an HLB value of 1.5-9. By way of an example, polysorbate 80 has an HLB value of 15 and is therefore a hydrophilic surfactant, whereas sorbitan trioleate has an HLB value of 1.8 and is therefore a lipophilic surfactant. The HLB of mixed surfactants is calculated according to their relative weightings (by volume) e.g. a 1:1 mixture by volume of polysorbate 80 and sorbitan trioleate has a HLB of 8.4.

In one embodiment, the composition includes a hydrophilic non-ionic surfactant in an amount of from about 5% to about 40% by weight of the composition, optionally from about 10% to about 30% by weight of the composition. Preferably, the composition includes a hydrophilic non-ionic surfactant in an amount of from about 10% to about 20% by weight of the composition, such as from about 10% to about 15% by weight of the composition.

The hydrophilic non-ionic surfactant may be a polyethylene glycol ester of a vegetable oil containing at least 20 moles of ethylene oxide groups/mole of glyceride. Suitable polyethylene glycol esters are typically selected from polyoxyethylene castor oil derivatives (e.g. PEG 20, 30, 35, 38, 40, 50 and 60 castor oil or PEG 20, 25, 30, 40, 45, 50, 60 and 80 hydrogenated castor oil), PEG 20 and 60 corn glycerides, PEG 20 and 60 almond glycerides, PEG 40 palm kernel oil, sodium laurate sulfate, sucrose esters (e.g. sucrose stearate, sucrose distearate, sucrose cocoate or sucrose monolaurate), PEG cocoglyceride, PEG 8 caprylocaprate, polyglyceryl esters and linolenamide DEA.

In certain embodiments, the hydrophilic non-ionic surfactant may be a mixture of acrylamide acryloyldimethyl taurate copolymer, isohexadecane and polysorbate 80, such as that available under the trade name SEPINEO P600. The hydrophilic non-ionic surfactant may be an alkylpolyglucoside, such as that available under the trade name SEPINEO SE68.

In one embodiment, the composition includes a lipophilic non-ionic surfactant in an amount of from about 0.1% to about 5% by weight of the composition. In other embodiments, the lipophilic non-ionic surfactant may be present in an amount of from about 0.1% to about 40% by weight of the composition. Surfactants are generally irritants, and so it is preferred to use only low levels of certain surfactants. However, some lipophilic non-ionic surfactants, such as monoglyceride esters, are less irritative and so can be present in higher amounts without causing significant levels of skin irritation.

The lipophilic non-ionic surfactant may be selected from monoglyceride esters of C₆₋₂₂ fatty acids (e.g. glyceryl monocaprylate, glyceryl monocaprate, glyceryl monostearate, glyceryl monobehenate), diglyceride esters of C₆₋₂₂ fatty acids (e.g. glyceryl dilaurate), mono- and diglyceride esters of C₆₋₂₂ fatty acids (e.g. caprylic/capric mono- and diglyceride, glyceryl mono- and diricinoleate), propylene glycol esters of C₆₋₂₂ fatty esters (e.g. propylene glycol monocaprylate, propylene glycol monolaurate), dialkylene glycol monoalkyl ethers (e.g. diethylene glycol monoethyl ether), polyglyceryl C₆₋₂₂ fatty acid esters (e.g. polyglyceryl-3-diisostearate), polyethylene glycol esters of a triglyceride/vegetable oil containing 4 to 8 moles of ethylene oxide groups/mole of glyceride (e.g. PEG-6 corn oil, PEG-6 almond oil, PEG-6 apricot kernel oil, PEG-6 olive oil, PEG-6 peanut oil, PEG-6 palm kernel oil, hydrogenated palm kernel oil, PEG-6 triolein, PEG-8 corn oil), polysorbates (e.g. polysorbate 20, polysorbate 40, polysorbate 60, polysorbate 80).

### Keratinolytic Agents

In some embodiments, the composition includes a keratinolytic agent, such as an α-hydroxy acid or β-hydroxy acid. The use of a keratinolytic agent may improve penetration of the active substance, meaning that compositions comprising a keratinolytic agent are particularly useful for treating hyperkeratotic actinic keratosis.

Suitable keratinolytic agents for use in the compositions of the invention include retinoids, adapalene, tars, shale oil, allantoin, aluminium oxide, azelaic acid, benzoyl peroxide, lactic acid, salicylic acid, alcali and alkali earth sulfide, monochloroacetic acid, urea, and resorcin. Particular retinoids that may be suitable include retinol, retinaldehyde, retinoic acid, isotretinoin, adapalinen and tazarotene. Further keratinolytic agents include ammonium glycolate, ammonium lactate, betaine salicylate, calcium lactate, calcium thioglycolate, glycolic acid, lactic acid, phenol, potassium lactate and sodium lactate.

In one embodiment, the composition includes an α-hydroxy acid selected from glycolic acid, lactic acid, mandelic acid, malic acid, citric acid and tartaric acid. In another embodiment, the composition includes a β-hydroxy acid such as salicylic acid. Preferably, the keratinolytic agent is salicylic acid.

The keratinolytic agent (e.g. α-hydroxy acid or β-hydroxy acid) may be present in an amount of from about 0.1% to about 20% by weight of the composition, e.g. about 0.5%, 1.0%, 2.5%, 5.0%, 7.5%, 10%, 15% or 20% by weight of the composition. Preferably, the composition includes salicylic acid in an amount of from about 0.1% to about 20% by weight of the composition, e.g. about 0.5%, 1.0%, 2.5%, 5.0%, 7.5%, 10%, 15% or 20% by weight of the composition.

### Exclusions

In some embodiments, the invention does not encompass any substantially anhydrous topical gel compositions which consist essentially of ingenol-3-angelate, benzyl alcohol, cyclomethicone, isopropyl myristate and Elastomer 10 (Dow Corning® ST-Elastomer 10), if any ingenol-3-angelate therein is present in suspended form.

In some embodiments, the invention does not encompass any substantially anhydrous topical gel compositions which consist essentially of:
(i) 0.08% by weight ingenol-3-angelate, 99.92% by weight Miglyol 810 (mixture of caprylic and capric triglycerides);
(ii) 0.1% by weight ingenol-3-angelate, 1% by weight benzyl alcohol, 9.5% by weight cyclomethicone, 9.5% by weight isopropyl myristate, and Elastomer 10 (Dow Corning® ST-Elastomer 10);
(iii) 0.1% by weight ingenol-3-angelate, 1% by weight benzyl alcohol, 69.9% by weight glycerol, 26.5% by weight PEG 400, 1.0% by weight hydroxypropyl cellulose and 1.5% by weight carbomer-934; or
(iv) 0.1% by weight ingenol-3-angelate, 1% by weight benzyl alcohol, 69.9% by weight glycerol, 26.0% by weight PEG 400, 1.5% by weight hydroxypropyl cellulose and 1.5% by weight carbomer-934,
   if any ingenol-3-angelate therein is present in suspended form.

### Optional Disclaimers

In some embodiments, the composition does not consist essentially of:
(i) ingenol-3-angelate, benzyl alcohol, polyoxypropylene-15-stearyl ether, liquid paraffin and Aerosil 200P; or
(ii) ingenol-3-angelate, benzyl alcohol, polyoxypropylene-15-stearyl ether, isopropanol, liquid paraffin and Aerosil 200P,
   if any ingenol-3-angelate therein is present in suspended form.

In some embodiments, the composition does not consist essentially of:
(i) ingenol-3-angelate, benzyl alcohol in an amount of 0.9% by weight of the composition, polyoxypropylene-15-stearyl ether in an amount of 10% by weight of the composition, liquid paraffin in an amount of 84.05% by weight of the composition and Aerosil 200P in an amount of 5% by weight of the composition; or
(ii) ingenol-3-angelate, benzyl alcohol in an amount of 0.9% by weight of the composition, polyoxypropylene-15-stearyl ether in an amount of 10% by weight of the composition, isopropanol in an amount of 20% by weight of the composition, liquid paraffin in an amount of 64.05% by weight of the composition and Aerosil 200P in an amount of 5% by weight of the composition,
   if any ingenol-3-angelate therein is present in suspended form.

In some embodiments, if the composition comprises ingenol-3-angelate in an amount of 0.05% by weight of the composition, then the composition does not consist essentially of:
(i) ingenol-3-angelate in an amount of 0.05% by weight of the composition, benzyl alcohol in an amount of 0.9% by weight of the composition, polyoxypropylene-15-stearyl ether in an amount of 10% by weight of the composition, liquid paraffin in an amount of 84.05% by weight of the composition and Aerosil 200P in an amount of 5% by weight of the composition; or
(ii) ingenol-3-angelate in an amount of 0.05% by weight of the composition, benzyl alcohol in an amount of 0.9% by weight of the composition, polyoxypropylene-15-stearyl ether in an amount of 10% by weight of the composition, isopropanol in an amount of 20% by weight of the composition, liquid paraffin in an amount of 64.05% by weight of the composition and Aerosil 200P in an amount of 5% by weight of the composition,
   if any ingenol-3-angelate therein is present in suspended form.

In some embodiments, the composition is not a substantially anhydrous pharmaceutical gel composition for cutaneous application comprising ingenol-3-angelate in dissolved or solubilized form, an acidifying compound, a co-solvent, a viscosity-increasing ingredient, a non-aqueous carrier and a solvent mixture of
(a) a hydrophilic non-ionic surfactant;
(b) a lipophilic non-ionic co-surfactant; and
(c) an oil,
   if any ingenol-3-angelate therein is present in suspended form.

In some embodiments, the composition does not consist essentially of:
(i) ingenol-3-angelate, benzyl alcohol, Miglyol 812, Akoline MCM, Cremophor RH40, liquid paraffin, white soft paraffin, hard paraffin and lactic acid;
(ii) ingenol-3-angelate, benzyl alcohol, N-methylpyrrolidone, Miglyol 812, Akoline MCM, Cremophor RH40, liquid paraffin, microcrystalline wax and lactic acid;
(iii) ingenol-3-angelate, benzyl alcohol, Miglyol 812, Akoline MCM, Cremophor RH40, liquid paraffin, microcrystalline wax and citric acid;
(iv) ingenol-3-angelate, benzyl alcohol, Miglyol 812, Akoline MCM, Cremophor RH40, liquid paraffin, white soft paraffin and salicylic acid; or
(v) ingenol-3-angelate, benzyl alcohol, Miglyol 812, Akoline MCM, Cremophor RH40, liquid paraffin, white soft paraffin and tartaric acid,
   if any ingenol-3-angelate therein is present in suspended form.

In some embodiments, if the composition comprises ingenol-3-angelate in an amount of 0.05% by weight of the composition, then the composition does not consist essentially of:
(i) ingenol-3-angelate, benzyl alcohol, Miglyol 812, Akoline MCM, Cremophor RH40, liquid paraffin, white soft paraffin, hard paraffin and lactic acid;
(ii) ingenol-3-angelate, benzyl alcohol, N-methylpyrrolidone, Miglyol 812, Akoline MCM, Cremophor RH40, liquid paraffin, microcrystalline wax and lactic acid;
(iii) ingenol-3-angelate, benzyl alcohol, Miglyol 812, Akoline MCM, Cremophor RH40, liquid paraffin, microcrystalline wax and citric acid;
(iv) ingenol-3-angelate, benzyl alcohol, Miglyol 812, Akoline MCM, Cremophor RH40, liquid paraffin, white soft paraffin and salicylic acid; or
(v) ingenol-3-angelate, benzyl alcohol, Miglyol 812, Akoline MCM, Cremophor RH40, liquid paraffin, white soft paraffin and tartaric acid,
   if any ingenol-3-angelate therein is present in suspended form.

In some embodiments, the composition does not consist essentially of:
(i) ingenol-3-angelate in an amount of 0.05% by weight of the composition, benzyl alcohol in an amount of 0.9% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 48.05-48.55% by weight of the composition, white soft paraffin in an amount of 40% by weight of the composition, hard paraffin in an amount of 0.5-1% by weight of the composition and lactic acid in an amount of 0.5% by weight of the composition;
(ii) ingenol-3-angelate in an amount of 0.05% by weight of the composition, benzyl alcohol in an amount of 0.9% by weight of the composition, N-methylpyrrolidone in an amount of 5% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 83.05% by weight of the composition, microcrystalline wax in an amount of 5% by weight of the composition and lactic acid in an amount of 1% by weight of the composition;
(iii) ingenol-3-angelate in an amount of 0.05% by weight of the composition, benzyl alcohol in an amount of 0.9% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 78.85% by weight of the composition, microcrystalline wax in an amount of 10% by weight of the composition and citric acid in an amount of 0.2% by weight of the composition;
(iv) ingenol-3-angelate in an amount of 0.05% by weight of the composition, benzyl alcohol in an amount of 0.9% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 73.55% by weight of the composition, microcrystalline wax in an amount of 15% by weight of the composition and citric acid in an amount of 0.5% by weight of the composition;
(v) ingenol-3-angelate in an amount of 0.05% by weight of the composition, benzyl alcohol in an amount of 9% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 48.55% by weight of the composition, white soft paraffin in an amount of 40% by weight of the composition and salicylic acid in an amount of 0.5% by weight of the composition; or
(vi) ingenol-3-angelate in an amount of 0.05% by weight of the composition, benzyl alcohol in an amount of 9% by weight of the composition, Miglyol 812 in an amount of 2.5% by weight of the composition, Akoline MCM in an amount of 2.7% by weight of the composition, Cremophor RH40 in an amount of 4.8% by weight of the composition, liquid paraffin in an amount of 48.55% by weight of the composition, white soft paraffin in an amount of 40% by weight of the composition and tartaric acid in an amount of 0.5% by weight of the composition,
   if any ingenol-3-angelate therein is present in suspended form.

In some embodiments, the invention does not encompass a substantially anhydrous topical gel compositions which consists essentially of:
(i) ingenol-3-angelate and Miglyol gel;
(ii) ingenol-3-angelate, benzyl alcohol, cyclomethicone, isopropyl myristate and Elastomer 10 (Dow Corning® ST-Elastomer 10); or
(iii) ingenol-3-angelate, benzyl alcohol, glycerol, PEG 400, hydroxypropyl cellulose and carbomer-934,
   if any ingenol-3-angelate therein is present in suspended form.

In some embodiments, the invention does not encompass compositions in which the ingenol-3-angelate is present in an amount of from 0.08% to 0.1% by weight of the composition when the composition consists essentially of:
(i) ingenol-3-angelate and Miglyol gel;
(ii) ingenol-3-angelate, benzyl alcohol, cyclomethicone, isopropyl myristate and Elastomer 10 (Dow Corning® ST-Elastomer 10); or
(iii) ingenol-3-angelate, benzyl alcohol, glycerol, PEG 400, hydroxypropyl cellulose and carbomer-934,
   if any ingenol-3-angelate therein is present in suspended form.

In most embodiments, the ingenol-3-angelate is not present at more than 0.06% by weight of the composition i.e. ingenol-3-angelate is present in the composition in an amount of 0.06% or less by weight of the composition, if any ingenol-3-angelate therein is present in suspended form.

In some embodiments, the ingenol-3-angelate is not present in the composition in an amount of from 0.08% to 0.1% by weight of the composition, if any ingenol-3-angelate therein is present in suspended form.

In some embodiments, the composition includes one or more silicones but does not include isopropyl myristate, if any ingenol-3-angelate therein is present in suspended form.

### Stability of the Compositions

The inventors have found that compositions of the invention exhibit very favorable stability properties.

In some embodiments, the composition is chemically stable, where chemically stable (or chemical stability) means that less than 10% of the ingenol-3-angelate degrades when the gel is stored for 2 years at 25° C. In some preferred embodiments, less than 6% of the ingenol-3-angelate degrades over a storage period of 2 years. An approximation of this chemical stability can be obtained by subjecting the composition to stability studies at 25° C. for 6 months: if less than about 2.5% of the ingenol-3-angelate has degraded after 6 months at 25° C. then a shelf-life of 2 years at room temperature is expected, i.e. less than 10% of the ingenol-3-angelate will be expected to degrade over a storage period of 2 years at 25° C. These studies are carried out according to ICH Humidity Guidelines, at conditions of 25° C.±2°/60% RH±5%, in hermetically sealed containers.

Preferred chemically stable gels include, after storage for 2 years at 25° C., less than 5% by weight of total ingenanes in the composition are ‘A’ and/or ‘B’. Thus, if the total amount of ‘A’ and ‘B’ exceeds 5% by weight of the total ingenanes, the gel's shelf-life is not ideal.

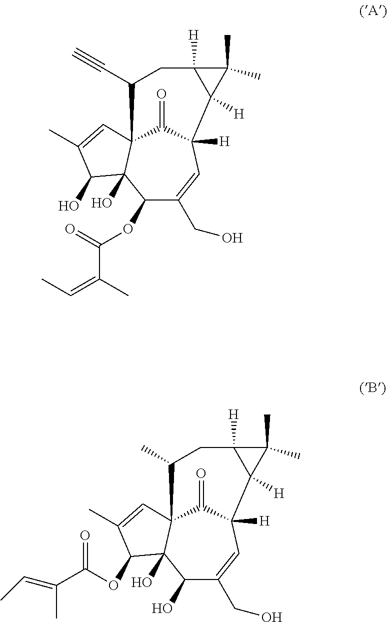

In some embodiments, the composition is physically stable, where physically stable (or physical stability) means that the composition retains its macroscopic and microscopic appearance over the shelf-life of the product, e.g. any dissolved ingenol-3-angelate does not precipitate from the solvent phase.

In some embodiments, the composition is chemically stable and physically stable.

### Penetration and Permeation of the Compositions

The inventors have found that compositions of the invention can exhibit very favorable skin penetration characteristics. Skin penetration means the flux of the active ingredient into the different layers of the skin, i.e. the stratum corneum, epidermis and dermis, after application of the gel to the skin.

In some embodiments, the compositions exhibit greater flux, according to the in vitro diffusion test, of ingenol-3-angelate into the stratum corneum, epidermis and dermis after application of the gel to skin than does a reference gel of ingenol-3-angelate; wherein the reference gel (a) has the same strength of ingenol-3-angelate as the topical gel composition, (b) consists essentially of ingenol-3-angelate, benzyl alcohol, isopropyl alcohol in an amount of 30% by weight of the formulation, hydroxyethyl cellulose in an amount of 1.5% by weight of the formulation and citrate buffer solution in an amount of 67.55% by weight of the formulation, and (c) is prepared by mixing ingenol-3-angelate with benzyl alcohol, and then adding the remaining components to the mixture of ingenol-3-angelate and benzyl alcohol in the order of: isopropyl alcohol, a citrate buffer solution formed from citric acid in an amount of 0.56% by weight of the formulation, sodium citrate dihydrate in an amount of 0.14% by weight of the formulation and water in an amount of 66.85% by weight of the formulation, and then hydroxyethyl cellulose to form the reference gel.

The in vitro diffusion test is performed as follows:
(a) Full thickness skin from pig ears should be used. The ears should be frozen at −18° C. before use, and placed in a refrigerator (5±3° C.) for slow defrosting on the day prior to use in the experiment. On the day of the experiment, the hairs should be removed using a veterinary hair trimmer. The skin should then be cleaned for subcutaneous fat using a scalpel, and two pieces of skin should be cut from each ear and mounted on Franz diffusion cells in a balanced order. Flow-through Franz-type diffusion cells (e.g. with an available diffusion area of 3.14 cm² and receptor volumes ranging from 11.1 to 12.6 ml) are used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68, with the specific volume being measured and registered for each cell. A magnetic bar should be placed in the receptor compartment of each cell.
(b) After mounting the skin, physiological saline (35° C.) should be filled into each receptor chamber for hydration of the skin. The cells should then be placed in a thermally controlled water bath which is placed on a magnetic stirrer set at 400 rpm. The circulating water in the water baths should be kept at 35±1° C. resulting in a temperature of about 32° C. on the skin surface.
(c) After half an hour the saline should be replaced by receptor medium, 0.04 M isotonic phosphate buffer, pH 7.4 (35° C.), containing 4% bovine serum albumin, and left for hydration for another hour. The inlet and outlet ports of the receptor chamber should be connected to stainless steel HPLC tubing. The cells should be connected to a 12-channel peristaltic pump, and the receptor fluid should be pumped continuously through each cell and collected in vials placed at a fraction collector. A controller can be used to program the duration of each fraction independently. Sink conditions should be maintained at all times during the period of the study, i.e. the concentration of the active compounds in the receptor medium should be below 10% of the solubility of the compounds in the medium.
(d) The in vitro diffusion test should be carried out in 6 replicates (i.e. n=6). Each test composition should be applied to the skin membrane at 0 hours in an intended dose of 4 mg/cm². A glass spatula should be used for the application, and the residual amount of the composition should be determined to give the amount of composition actually applied to the skin.
(e) The in vitro diffusion test should be allowed to proceed for 21 hours.
(f) About 6 ml of the receptor fluid should be sampled from each cell every third hour until 21 hours post-application. The sample collection of the first 45 minutes should be discarded due to the lag time of the system. The recipient fluid remaining in the diffusion cell at the end of the study should correspond to the 21 hour sample.
(g) The stratum corneum should be collected by tape stripping 10 times using D-Squame® tape (diameter 22 mm, CuDerm Corp., Dallas, Tex., USA). Each tape strip should be applied to the test area using a standard pressure for 5 seconds and removed from the test area in one gentle, continuous move. For each repeated strip, the direction of tearing off should be varied. The viable epidermis and dermis can then be sampled from the skin by taking a full biopsy of 3.14 cm² of the applied area for analysis. The skin surrounding the test area should be discarded.
(h) The concentration of ingenol-3-angelate in the samples should then be determined by LC-MS/MS.

If the total amount of ingenol-3-angelate in the stratum corneum, epidermis and dermis as a percentage of the applied dose, as determined in step (h), is higher than for the reference gel (e.g. PICATO® at the same strength of ingenol-3-angelate as the topical gel composition), then the composition is said to exhibit more penetration (i.e. greater flux of the active ingredient into the stratum corneum, epidermis and dermis after application of the gel to the skin).

In some embodiments, the composition exhibits less penetration than the reference gel according to this assay, i.e. the total amount of ingenol-3-angelate in the stratum corneum, epidermis and dermis (combined) as a percentage of the applied dose, as determined in step (h), is lower than for the reference gel (e.g. PICATO® at the same strength of ingenol-3-angelate as the topical gel composition).

Skin permeation means the flux of the active ingredient through the skin into the systemic circulation or, in case of in vitro studies, the receptor fluid of the Franz cell apparatus used in the experiment, after application of the gel to the skin. In some embodiments, the composition exhibits less permeation than does the reference gel according to this assay, where less potent permeation means that the amount of ingenol-3-angelate in the receptor fluid as a percentage of the applied dose, as determined in step (h), is lower than for the reference gel (e.g. PICATO® at the same strength of ingenol-3-angelate as the topical gel composition). This may be desirable to avoid unnecessary levels of systemic ingenol-3-angelate.

### Processes

In some embodiments, gel compositions of the present invention are made by mixing solid ingenol-3-angelate with a liquid non-aqueous carrier, and then adding a solid gelling agent to form the gel.

In other embodiments, the gel compositions may be made by mixing solid ingenol-3-angelate with a solid non-aqueous carrier, and then adding a liquid to form the gel.

Thus it is preferred to form a suspension (solid or liquid) of the ingenol-3-angelate, and then to add enough gelling agent to form a gel having the desired characteristics. Less preferably, the gelling agent and ingenol-3-angelate are mixed prior to adding to a non-aqueous solid carrier.

### Medical Treatments and Uses

The invention also provides a method for treating a dermal disease or condition, comprising topical administration of a gel of the invention to a mammal Topical administration means that the compositions are applied cutaneously i.e. to the external skin on the body.

The invention also provides a gel of the invention for use in treating a dermal disease or condition.

The invention also provides the use of ingenol-3-angelate and a non-aqueous carrier in the manufacture of a gel medicament for treating a dermal disease or condition.

The uses and methods are useful for the topical treatment of dermal diseases or conditions including actinic keratosis, seborrheic keratosis, skin cancer, warts, keloids, scars, photoaged or photodamaged skin, and acne. In particular, the uses and methods are particularly useful for the topical treatment of actinic keratosis. The uses and methods may, for instance, be useful for the topical treatment of hyperkeratotic actinic keratosis.

The uses and methods may be used for the topical treatment of skin cancers such as non-melanoma skin cancer, malignant melanoma, Merkel cell carcinoma, squamous cell carcinoma or basal cell carcinoma (including superficial basal cell carcinoma and nodular basal cell carcinoma).

The uses and methods may be used for the topical treatment of warts, e.g. human papilloma virus (HPV) infections on the skin, genitals and mouth.

The uses and methods may be used for the topical treatment of photodamaged skin such as fine lines, wrinkles and UV-ageing. UV-ageing is often manifested by an increase in the epidermal thickness or epidermal atrophy, most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting in a leathery appearance, skin fragility and irregular pigmentation, loss of tone and elasticity, as well as wrinkling, dryness, sunspots and deep furrow formation.

The uses and methods may be useful for reducing or minimizing scar tissue or improving cosmesis or functional outcome in a wound. For instance, the uses and methods may be useful for improving functional outcome in a wound which is cutaneous, chronic or diabetes associated, e.g. when the wound includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant, immune response conditions, oral wounds, stomach or intestinal wounds, damaged cartilage or bone, amputation sides and corneal lesions.

Therefore, in some embodiments, the uses and methods are cosmetic.

Typically, the uses and methods are lesion specific, i.e. they are focused on a lesion being treated and do not extend to any larger degree to the surrounding skin. In other embodiments, however, the uses and methods can extend to a larger area than the lesions, and this can usefully lead to treatment of emerging lesions or sub-surface pre-lesions. Also, it can be convenient to apply a gel to an area which includes several lesions, rather than applying it to each individual lesion in that area. The lesions could be of any size (i.e. surface area), e.g. greater than about 30 000 mm², greater than about 20 000 mm², greater than about 10 000 mm², greater than about 5000 mm², greater than about 1000 mm², greater than about 500 mm², greater than about 250 mm², or greater than about 150 mm². Typically, the lesion size is about 30 000 mm², about 20 000 mm², about 10 000 mm², about 5000 mm², about 1000 mm², about 500 mm², about 250 mm², about 150 mm², about 100 mm², about 75 mm², about 50 mm², about 25 mm² or about 10 mm².

In the treatment of, for example, actinic keratosis on the face and/or scalp of a subject, a gel composition of the invention may be applied on the face and scalp to the affected skin area (treatment area) once a day for 3 consecutive days. In the treatment of, for example, actinic keratosis on the trunk and/or extremities of a subject, a gel composition of the invention may be applied on the trunk and extremities to the affected skin area (treatment area) once a day for 2 consecutive days Immediately following application of a gel to the treatment area, subjects should wash their hands.

The gel compositions of the invention are typically packaged in hermetically sealed containers, e.g. a unit dose tube. A unit dose tube would typically contain about 0.5 g of gel. Preferably, one unit dose tube (tube with screw cap or individual packets) may be used for one treatment area.

### DEFINITIONS

The term “substantially anhydrous” means that the content of free water in the composition is less than about 2% by weight, preferably less than about 1% by weight, such as less than about 0.5% by weight, of the composition.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

### MODES FOR CARRYING OUT THE INVENTION

The invention is further illustrated by the followings examples. It will be appreciated that the examples are for illustrative purposes only and are not intended to limit the invention as described above. Modification of detail may be made without departing from the scope of the invention.

### Example A

### Preparation of Compositions of the Invention

The following compositions were prepared:

### Composition Series 11

### 03A

PEP005 0.5 mg/g
Paraffin, liquid 799.5 mg/g
Microcrystalline wax 200 mg/g
   Prepared as follows. Make a PEP005 suspension by grinding purified crystals in 10 ml liquid paraffin to obtain a homogeneous suspension (and keep 5 ml of liquid paraffin for rinsing). Add the rest of the excipients in a steel container and melt them together. Cool this melted mixture to 30° C. while mixing. Add this microcrystalline wax gel stepwise to the PEP005 suspension under careful mixing.

### 04A

PEP005 0.5 mg/g
Dow Corning® ST-Elastomer 10 750 mg/g
Dow Corning® ST cyclomethicone 249.5 mg/g
   Make a PEP005 suspension by grinding purified crystals in cyclomethicone (50 mg/g) to obtain a homogeneous suspension. Pour the rest of the cyclomethicone into the suspension under continuous mixing, then blend in the ST-Elastomer to make the final gel suspension.

### 05A

PEP005 0.5 mg/g
Paraffin, liquid 939.5
Aerosil 200P 60 mg/g
   Make a PEP005 suspension by grinding purified crystals in liquid paraffin (50 mg/g) to obtain a homogeneous suspension. Make a gel of the rest of liquid paraffin and the Aerosil 200P, by mixing them together. Add this paraffin/Aerosil gel to the PEP005 suspension stepwise under mixing until a homogeneous suspension is obtained.

### Example B

### Chemical Stability Studies

A number of compositions of the invention were tested for chemical stability. This testing required extraction of ingenol-3-angelate from the composition by dissolution in a solvent mixture of acetonitrile and phosphoric acid. Following extraction, organic impurities were identified using reversed phase HPLC with UV detection at 220 nm. The following compositions from Example A were found to be stable after 6 months at 25° C., indicating that less than 10% of the ingenol-3-angelate would be expected to degrade over a storage period of 2 years at room temperature (25° C.):
Composition series 11, formulations 03A and 04A

### Example C

### Skin Penetration and Permeation Studies

To investigate the skin penetration and permeation of ingenol-3-angelate from compositions of the invention, an in vitro skin diffusion test was conducted.

Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Flow-through Franz-type diffusion cells with an available diffusion area of 3.14 cm² and receptor volumes ranging from 11.1 to 12.6 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After half an hour the saline was replaced by receptor medium, 0.04 M isotonic phosphate buffer, pH 7.4 (35° C.), containing 4% bovine serum albumin and left for hydration another hour. The inlet and outlet ports of the receptor chamber were connected to stainless steel HPLC tubing. The cells were connected to a 12-channel peristaltic pump, and the receptor fluid was pumped continuously through each cell and collected in vials placed at a fraction collector. A controller was used to program independently the duration of each fraction. Sink conditions were maintained at all times during the period of the study, i.e. the concentration of the active compounds in the receptor medium was below 10% of the solubility of the compounds in the medium.

The in vitro skin penetration and permeation was tested in 6 replicates (i.e. n=6). Each test composition was applied to the skin membrane at 0 hours in an intended dose of 4 mg/cm². A glass spatula was used for the application, and the residual amount of the composition was determined so as to give the amount of the composition actually applied on the skin.

### Permeation and Penetration

The skin penetration and permeation experiment was allowed to proceed for 21 hours. Samples were then collected from the following compartments:

About 6 ml of the receptor fluid was sampled from each cell every third hour until 21 hours post application. The sample collection of the first 45 minutes was discarded due to the lag time of the system. The recipient fluid remaining in the diffusion cell at the end of the study corresponded to the 21 hour sample.

The stratum corneum was collected by tape stripping 10 times using D-Squame® tape (diameter 22 mm, CuDerm Corp., Dallas, Tex., USA). Each tape strip was applied to the test area using a standard pressure for 5 seconds and removed from the test area in one gentle, continuous move. For each repeated strip, the direction of tearing off was varied. The viable epidermis and dermis was then sampled from the skin by taking a full biopsy of 3.14 cm² of the applied area for analysis. The skin surrounding the test area was discarded.

The concentration of ingenol-3-angelate in the samples was determined by LC-MS/MS.

### Results

These studies allowed the amount of ingenol-3-angelate found in the stratum corneum, epidermis and dermis and receptor fluid to be calculated, as a percentage of the applied dose.

The following compositions from Example A exhibited more penetration than PICATO® at the same strength of ingenol-3-angelate by weight of the composition:
Composition series 11, formulation 03A

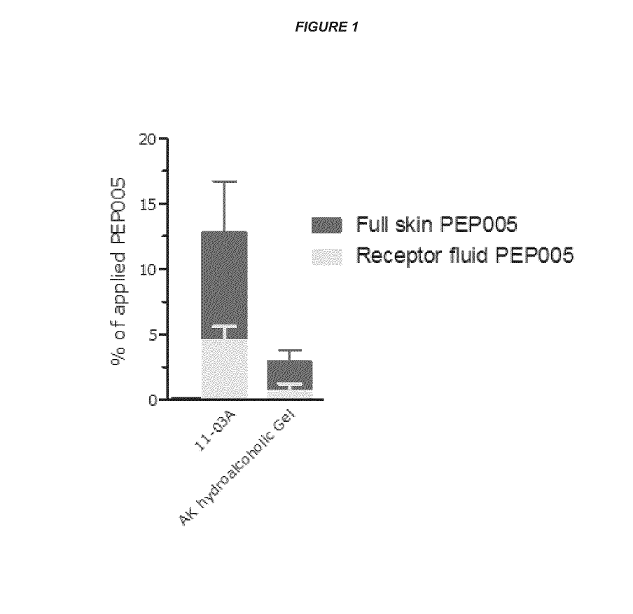

The data for composition series 11, formulation 03A are shown in FIG. 1, which show that the amount of ingenol-3-angelate found in the stratum corneum, epidermis and dermis after application of composition series 11, formulation 03A is significantly higher than the amount found in the stratum corneum, epidermis and dermis after application of PICATO® at the same strength of ingenol-3-angelate by weight of the composition.

It will be understood that the invention has been described by way of example only and modifications may be made whilst remaining within the scope and spirit of the invention.

1. A substantially anhydrous topical gel composition comprising ingenol-3-angelate as a suspension in a non-aqueous carrier.

2. The composition of claim 1, wherein the ingenol-3-angelate is present in an amount of about 0.0005%, 0.001%, 0.0025%, 0.005%, 0.01%, 0.015%, 0.025%, 0.05%, 0.075%, 0.1%, 0.125%, 0.15%, 0.2%, 0.25% or 0.5% by weight of the composition.

3. The composition of claim 2, wherein the ingenol-3-angelate is present in an amount of about 0.015% or 0.05% by weight of the composition.

4. The composition of claim 1, wherein the ingenol-3-angelate is present as a crystalline suspension.

5. The composition of claim 1, wherein the composition is supersaturated.

6. The composition of claim 1, wherein the composition is acidic.

7. The composition of claim 1, wherein the non-aqueous carrier is present in an amount of from about 40% to about 99.95% by weight of the composition.

8. The composition of claim 1, wherein the composition includes a viscosity-increasing ingredient.

9. The composition of claim 8, wherein the viscosity-increasing ingredient is present in an amount of from about 0.5% to about 50% by weight of the composition.

10. The composition of claim 1, wherein the composition includes a co-solvent.

11. The composition of claim 10, wherein the co-solvent is present in an amount of from about 0.5% to about 20% by weight of the composition.

12. The composition of claim 1, wherein the composition includes a penetration enhancer.

13. The composition of claim 12, wherein the penetration enhancer is present in an amount of from about 0.01% to about 20% by weight of the composition.

14. The composition of claim 1, wherein the composition includes an acidifying compound.

15. The composition of claim 14, wherein the acidifying compound is present in an amount of from about 0.5% to about 10% by weight of the composition.

16. The composition of claim 1, wherein the composition includes a silicone.

17. The composition of claim 16, wherein the silicone functions as a non-aqueous carrier or a viscosity-increasing ingredient.

18. The composition of claim 1, wherein the composition is chemically stable.

19. The composition of claim 1, wherein the composition is physically stable.

20. The composition of claim 1, wherein the composition exhibits more penetration than a reference gel of the same strength of ingenol-3-angelate, according to an in vitro diffusion test.

21. A method for making a composition of claim 1, comprising mixing ingenol-3-angelate, a non-aqueous carrier, and a gelling agent.

22. The method of claim 21, comprising mixing solid ingenol-3-angelate with a liquid non-aqueous carrier, and then adding a solid gelling agent to form a gel.

23. A method for treating a dermal disease or condition, comprising topical administration to a mammal of a composition of claim 1.

24. The method of claim 23, wherein the dermal disease or condition is actinic keratosis.
